(19) United States
(12) Patent Application Publication
(10) Pub. No.: US 2024/0065573 A1
(43) Pub. Date: Feb. 29, 2024

(54) SYSTEM AND METHOD OF DETERMINING/TRACKING USAGE CAPABILITY OF MEDICAL DEVICES

(71) Applicant: St. Jude Medical, Cardiology Division, Inc., St. Paul, MN (US)

(72) Inventors: Isaac Remer, Richfield, MN (US); Timothy S. Marass, Minneapolis, MN (US)

(21) Appl. No.: 18/237,137

(22) Filed: Aug. 23, 2023

Related U.S. Application Data
(60) Provisional application No. 63/400,465, filed on Aug. 24, 2022.

(51) Int. Cl. A61B 5/06

(52) U.S. Cl. CPC A61B 5/062; A61B 2560/0238; A61B 2560/028; A61B 2560/066

(57) ABSTRACT

A method of monitoring total usage capability of a medical device includes monitoring one or more device attributes during a procedure and detecting device manipulations during the procedure based on the one or more device attributes. The method further includes determining whether the medical device may be reprocessed for subsequent use based, at least in part, on the detected device manipulations.

### CROSS-REFERENCE TO RELATED APPLICATION

This application claims benefit of priority to U.S. Provisional Patent Application No. 63/400,465 filed on Aug. 24, 2022, the entire disclosure of which is hereby incorporated by reference.

### BACKGROUND

The present invention relates generally to medical devices and in particular to the reprocessing of medical devices for additional use.

In some instances, medical devices are single use while others can be reprocessed and used in multiple procedures. Typically, a determination of whether a medical device can be reused is based on attributes such as number of procedures performed or hours of use. However, while useful, these attributes do not take into account the type of use or particular stresses experienced by a medical device. For example, a medical device such as a catheter used in a single procedure may be subjected to various levels of mechanical strains and stresses. It would be beneficial to account for stresses experienced by a medical device in determining whether a medical device can be reprocessed and used in additional procedures.

### SUMMARY

According to one aspect, a method of monitoring total usage capability of a medical device includes monitoring one or more device attributes during a procedure and detecting device manipulations during the procedure based on the one or more device attributes. The method further includes determining whether the medical device may be reprocessed for subsequent use based, at least in part, on the detected device manipulations.

According to another aspect, a method of tracking device manipulations includes receiving location information from two or more sensors located on a medical device during a procedure and utilizing the location information from the two or more sensors to detect device manipulations. The method further includes storing the detected device manipulations.

According to another aspect, a data process and analysis system configured to receive inputs from one or more sensors associated with a medical device includes a processor and memory for storing instructions executed by the processor to: determine locations of the one or more sensors based on inputs received from the one or more sensors; detect device manipulations based on the determined locations of the one or more sensors; and determine whether the medical device may be reprocessed for subsequent use based, at least in part, on the detected device manipulations.

### DESCRIPTION OF THE DRAWINGS

FIG. 1 is a diagrammatic view of a medical system according to some embodiments.

FIG. 2 is a flowchart of a method of determining whether a medical device may be reprocessed based on total usage capability according to some embodiments.

FIG. 3 is a flowchart of a method of determining device manipulations based on location information of sensors located on the device according to some embodiments.

FIGS. 4A and 4B are isometric views of an electrode array catheter assembly in a flexed and unflexed state according to some embodiments.

FIG. 5 is an isometric view of a loop catheter assembly according to some embodiments.

FIG. 6 is a diagrammatic view of a standard venous transseptal procedure according to some embodiments.

FIG. 7 is a diagrammatic view of a retrograde procedure according to some embodiments.

### DETAILED DESCRIPTION

The present disclosure is directed to a system and method of determining whether a medical device may be reprocessed based on device attributes monitored during a procedure. For example, device attributes, including for example the location of sensors during a procedure, may be analyzed to detect device manipulations. A total usage capability of the device is updated based on the one or more device attributes and utilized to determine whether a device may be reprocessed for additional use or should be discarded.

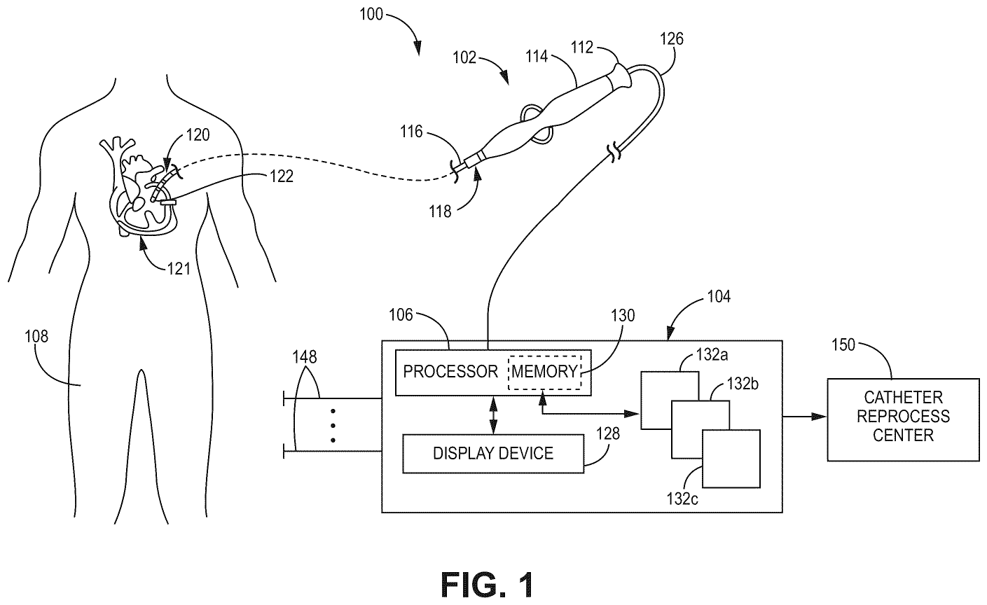

FIG. 1 is a diagrammatic view of a medical system 100 according to some embodiments. The system 100 comprises, among other components, a medical device 102 and a data collection and analysis systems 104 suitable for collecting EP data and other data as described herein from a subject and to generate outputs that include data displays, user interfaces, and other OT related features disclosed herein. In one embodiment, the medical device 102 is a catheter, which includes cable connector or interface 112, a handle 114, a shaft 116 having a proximal end 118 and a distal end 120, and at least one cable 126. In other embodiments, the medical device 102 may include other type of medical devices, including introducers, sheaths, guidewires, probes, etc. A plurality of electrodes 122 are located at the distal end 120 of the shaft 116 and may be utilized for a variety of functions, including impedance-based localization, EP mapping, and/or ablative treatments. The data collection and analysis system 104 includes a processing apparatus 106 having memory 130, and a display device 128. In some embodiments, the data collection and analysis system 104 is connected to a plurality of surface patch electrodes via connectors 148, which may be utilized to implement an impedance-based (or voltage-based) localization system for determining the location of the electrodes 122 within the patient's body 108 and/or heart 121. In other embodiments, additional sensors (e.g., magnetic-based sensors) may be utilized either alone or in conjunction with the impedance-based localization system to provide information regarding the location of one or more sensors. A magnetic-based localization system may generate a magnetic field over the patient's body 108. The location of a magnetic based sensor located at a distal end 120 of the device 102 can be determined based on the sensing/detection of the external magnetic field. In some embodiments, the processing apparatus 106 analyzes location data and/or EP mapping data to generate an anatomical model (e.g., a multi-dimensional model of the heart) that is presented on the display device 128. In some embodiments, the device 102 may further include one or more force sensors to measure the magnitude of force applied to, or applied by, the device 102, strain sensors, and/or one or more temperatures sensors.

In addition, data collection and analysis systems 104 is configured to receive measurements observed by the device 102. For example, data collection and analysis system 104 may receive voltages monitored by the plurality of electrodes 122 located at the distal end 120 of the device 102, both for impedance-based localization operations in conjunction with the surface patch electrodes 148 and electrocardiogram signals monitored by the plurality of electrodes 122.

The processing apparatus 106 may include one or more apparatus, devices, and machines for processing data, signals, and information, including by way of example a programmable processor, a computing device such as a computer, or multiple processors or computers. The apparatus can include, in addition to hardware, code that creates an execution environment for the computer program in question, e.g., code that constitutes processor firmware, a stack, a data management system, an operating system, one or more user interface systems, or a combination of one or more of them. Further, the processing apparatus 106 can include machine readable medium or other memory that includes one or more software modules 132a, 132b, and 132c for performing various functions. In some embodiments, software modules 132a, 132b, and/or 132c receive sensor signals from one or more sensors located on the device 102 and utilize the sensed signals to determine the location of each of the plurality of sensors within the body 108 as well as relative to one another. In some embodiments, software modules 132a, 132b, and/or 132c may further be capable of detecting device manipulations (e.g., flexing, opening/closing, bending) of the device 102. As described in more detail below, detected device manipulations can be utilized to determine whether a device can be reprocessed and utilized again, or should be discarded/retired. In some embodiments, this analysis may be performed locally by data collection and analysis system 104. In other embodiments, this analysis may be performed remotely at a reprocess center 150. In some embodiments, the data collection and analysis system 104 is configured to communicate electronically with the reprocess center 150. This may include communicating location information received with respect to sensors 122 located on the device, detected device manipulations, and/or other catheter attributes related to the deployment of a catheter within a patient's body 108.

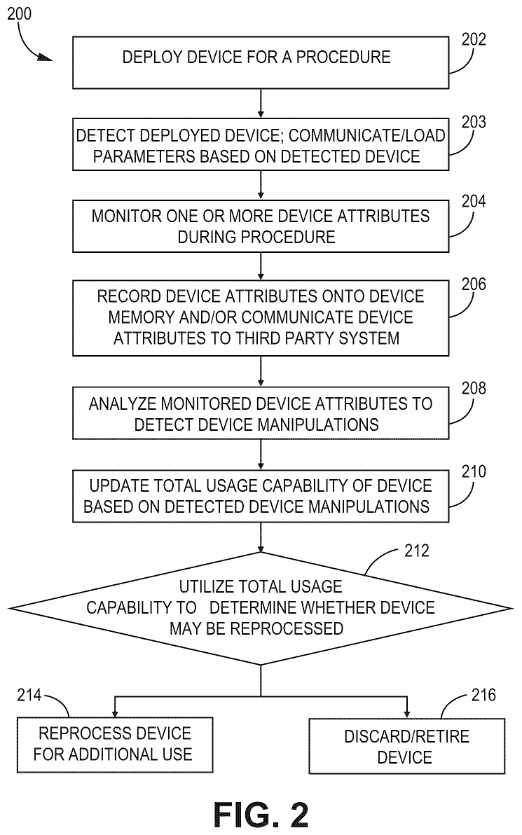

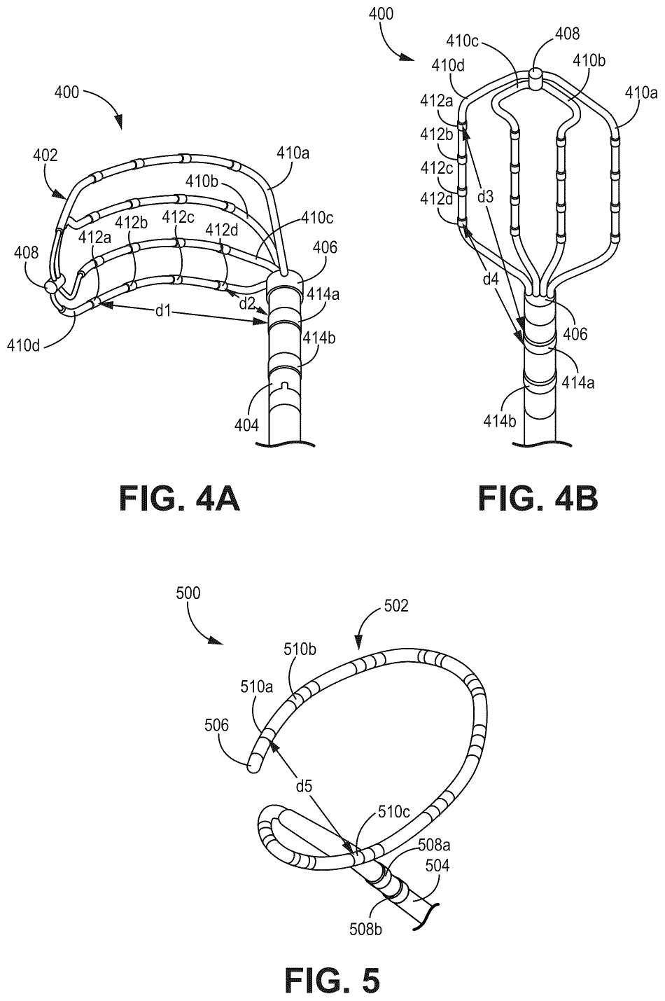

FIG. 2 is a flowchart of a method 200 of determining whether a medical device may be reprocessed based on total usage capability according to some embodiments. At step 202 the medical device is deployed for a procedure. As described above, medical devices may include introducers, probes, guidewires, and various types of catheters including diagnostic catheters and ablation catheters. In some embodiments, the distal end of the device may include various configurations and geometries (e.g., an end effector). For example, in some embodiments the device is a catheter that includes at a distal end a flexible grid-array of sensors (shown in FIGS. 4A and 4B). In other embodiments, the distal end of the device (e.g., catheter) includes a loop portion having one or more sensors (e.g., shown in FIG. 2). In other embodiments, various other types of devices may be deployed for various procedures.

At step 203, the deployed device is detected or otherwise identified, and the identification of the device is utilized to determine the criteria to be utilized to assess whether the device may be reprocessed. In some embodiments, a technician/user may manually enter identifying information regarding the deployed device, such as the specific model number of a particular device (e.g., HD grid catheter). In other embodiments, the system 100 may automatically detect or identify the deployed device. The identity of the deployed device is subsequently utilized to assess whether the device may be reprocessed following the procedure based on monitoring of one or more device attributes during the procedure. In some embodiments, the identity of the device determined at step 203 is utilized to load a set of parameters utilized to determine whether the device may be reprocessed. The set of parameters may be loaded locally onto the data collection and analysis systems 104 (shown in FIG. 1) and subsequently utilized or compared with data and/or attributes collected from the procedure to determine whether the device can be reprocessed. In other embodiments, identification of the device is communicated to the catheter reprocess center 150 (shown in FIG. 1), which accesses or otherwise retrieves the set of parameters utilized to determine whether the device can be reprocessed. In some embodiments, the set of parameters are stored locally (e.g., non-volatile memory) on the device itself (e.g., device 102). In some embodiments the set of parameters are downloaded or communicated to the data collection and analysis system 104 for subsequent analysis of whether a device may be reprocessed. In other embodiments, as described in more detail below, device attributes collected during the procedure may be stored to non-volatile memory associated with the device, wherein the determination of whether a device can be reprocessed is made based on the set of parameters stored locally on the device and the data attributes collected regarding the device during the procedure. In some embodiments, the set of parameters are pre-set and identify thresholds that determine whether a device can be reprocessed (e.g., number of procedures, number of device manipulations, etc.) and are unique to each model of device. In other embodiments, the set of parameters may be modified over time as data is collected from procedures.

At step 204, one or more device attributes are monitored during the procedure. In some embodiments, device attributes may include any information that may be collected during a given procedure. For example, this may include a duration of a procedure, number of treatments (e.g., activation events for ablative devices), electrode signal integrity, voltage and/or current carried by conductive wires to the electrodes, number of insertions/extractions, force measurements, temperature measurements, and/or location information associated with the device. More particularly, device attributes to be monitored are those attributes that can be measured and that are correlated with life of the device (referred to herein as the total usage capability of the device). For example, time of use is an attribute that can be easily recorded and has an impact on the total usage capability of the device. In this case, duration of use reduces the remaining total usage capability of the device. Likewise, delivery of ablation therapy can be monitored and is correlated with total usage capability of the device. For example, different ablation therapies may apply different amounts of voltage and/or current to the electrodes. In addition, location information can be monitored and can be utilized to detect device manipulations (i.e., movement/deflections of the device, typically the distal end of the device). Various types of location-based systems may be utilized to determine the location of sensors within space (as well as relative to one another). As described in FIG. 1, above, software modules 132a, 132b, and/or 132c may receive sensor signals from one or more sensors located on the device 102 and utilize the sensed signals to determine the location of each of the plurality of sensors within the body 108 as well as relative to one another. For example, magnetic-based sensors and/or impedance-based sensors may be utilized to determine the location of individual sensors within the system. In other embodiments, other types of localization systems may be utilized including fiber-optic based location systems. As described in more detail below, in some embodiments, location information is subsequently utilized to detect device manipulations.

At step 206, the one or more device attributes measured at step 204 are recorded or otherwise saved. In some embodiments, the monitored device attributes are stored on non-volatile memory local to the device itself. A benefit of this approach is that the monitored device attributes are stored on the device itself and do not need to be communicated. The device is sent to the reprocess center 150 (shown in FIG. 1) and the monitored device attributes are retrieved from the device and analyzed to determine whether the device can be reprocessed. In other embodiments, the monitored device attributes are stored on the data collection and analysis system 104. In some embodiments, the monitored device attributes may be analyzed locally by the data collection and analysis system 104, with the results of the analysis either stored onto the non-volatile memory of the device itself or communicated electronically to the reprocess center 150. A benefit of this latter approach is that the monitored device attributes may include large file sizes, which may be expensive to store locally on the device and/or communicate to the reprocess center 150. By analyzing the device attributes locally, only relevant data must be stored to the device and/or communicated. However, in other embodiments the monitored device attributes may be communicated electronically to reprocess center 150 or may be saved in their entirety onto non-volatile memory associated with the device and retrieved when the device is received by the reprocess center 150. In still other embodiments, the monitored device attributes may be uploaded to a cloud-based memory system that is accessible by the reprocess center 150.

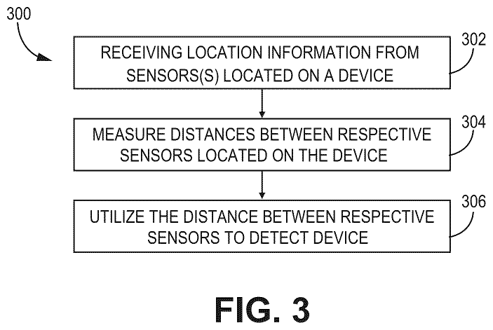

At step 208, device attributes are analyzed to detect device manipulations. Having determined the location of the sensors located on the device 102 within the body 108, the relative location of the sensors to one another are utilized to determine device manipulations, such as expanding/contracting, flexing, bending, or opening/closing of the device. Device manipulations may provide information about a local geometry (e.g., the distal end of the device), geometry of an end effector (e.g., expanded state or contracted state) or an overall geometry of the device. As described above, in some embodiments analysis of device attributes are performed locally by the data collection and analysis system 104 while in other embodiments device attributes are communicated to the reprocess center 150 for analysis. In some embodiments, device manipulations refer to events that put strain or stress onto the device. For example, bending or stressing of the device—particularly the distal end of the device—can result in fractures or cracks in the device. While some of these may be detected via visual inspection, some may be too small or in places that make visual inspection difficult. In some embodiments, location of the plurality of sensors carried by the device may be utilized to detect device manipulations. For example, as described with respect to FIG. 1, software modules 132a, 132b, and/or 132c included as part of the data collection and analysis system 104 may be utilized to determine the location of sensors within the body and relative to one another (e.g., impedance-based mapping, magnetic-based mapping, etc.). This is described in more detail in the flowchart shown in FIG. 3, which describes how the location of electrodes relative to one another may be utilized to detect device manipulations. Types of device manipulations include deflections, insertions/extractions, expansion/contraction of an end effector, bending of distal components, and/or bending/kinking of the device itself. In some embodiments, the types of sensors carried by the device determines the type of manipulations that can be detected. For example, bending of a grid array paddle located at a distal end of a device (e.g., shown in FIGS. 4a, 4b) may be detected based on location information retrieved from the plurality of sensors located on the grid array paddle. Movement of an end effector (e.g., grid or loop) relative to a sheath or introducer may be detected. For example, the sheath may include one or more location sensors and relative location information from the sheath sensor(s) and the end effector sensors may be utilized to determine a location of the end effector relative to the sheath (e.g., inside/outside of the sheath, distance from distal end of sheath). This information may be utilized to identify insertions/extractions of the end effector into/out of the sheath, or expansion/contraction of the end effector after the end effector has been positioned outside of the sheath. Deflections of a device (typically as a result of pull wire manipulation by an operator) may be detected based on sensors located in the handle 104 as well as based on location information retrieved from sensors located on the device. Bending or kinks in the catheter may be detected using shape fiber-optic sensor that extends along a length of the catheter. Device deflections, bending, or kinks may provide information about the overall geometry of the device. In general, device manipulations detected depend on the type of device being utilized and procedure being performed. For example, for a loop-type catheter device, manipulations to be detected include opening/closing of the loop, engagement of the loop with tissue, deployment/retraction of the loop from a distal end of an introducer, or bending along any length of the device.

In some embodiments, analysis may include monitoring the relative distance between one or more sensors over time to detect device manipulations. In some embodiments, identification of the device deployed at step 203 (and selection of parameters) is utilized in combination with the monitored sensor locations to detect and categorize device manipulations. For example, based on identification of the type of device utilized in a particular procedure, a change in distance between two sensors by a defined magnitude may indicate a particular manipulation of the device (e.g., bend). In some embodiments, parameters selected at step 203 based on the identification of the device utilized in the procedure includes a plurality of thresholds, wherein the thresholds are compared to various relationships between the sensors (e.g., relative distance between sensors) to detect various types and magnitudes of manipulations. For example, a first threshold distance between sensors may indicate a slight bend of a device, wherein a second threshold distance between sensors may indicate a greater bend of the device. In some embodiments, the magnitude of the manipulation is significant in determining the total usage capability of the device. For example, in some embodiments a weighting may be assigned to detected device manipulations based on the magnitude of the detected manipulation. In some embodiments, it may be beneficial to compare a relative distance between sensors to a plurality of thresholds to determine a particular magnitude of the device manipulation. In some embodiments, appropriate weighting is assigned based on the magnitude/severity of the device manipulation detected. A weighting may correspond to the stress and/or strain produced by the detected device manipulation. The detected device manipulations—along with the corresponding weighting—may be subsequently utilized to determine the usage associated with the monitored procedure. The usage associated with the procedure may then be utilized to determine the total usage capability of the device.

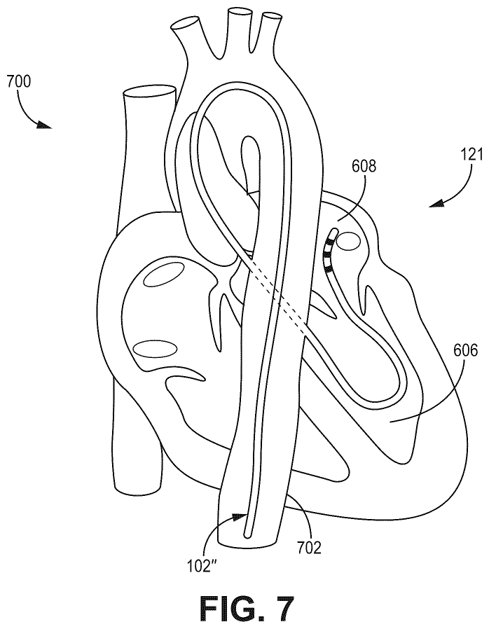

In other embodiments, analysis may include determining which procedure is being performed. As discussed above, a device 102 may include one or more location systems (e.g., impedance, magnetic, fiber-optic). Location information may be utilized to determine the type of procedure being performed. In some embodiments observed locations of the device during a procedure may be combined into a path that may be correlated to a procedure. In some embodiments, the observed locations are correlated to a location of the device relative to a lumen, organ, or anatomical model. In other embodiments, the location information is analyzed to provide information about the overall geometry of the device. The overall geometry of the device may vary during a procedure and/or between different procedures. In some embodiment, different procedures are identified by the overall geometry of the device when the device is positioned at the treatment location, while in other embodiments, different procedures are identified by comparing a sequence of overall geometries assumed by the device as the device is maneuvered to the treatment location. For example, FIGS. 6 and 7 respectively illustrate the geometry of catheters 102′(FIG. 6) and 102″ (FIG. 7) during a venous transseptal procedure 600 and a retrograde procedure 700 on the heart 121, which includes a right atrium 602, right ventricle 604, left ventricle 606, and left atrium 608. These procedure snapshots illustrate that when the distal end 120 of the device is positioned within the left atrium 608, the overall geometries assumed by the devices 102′, 102″ differ. Further, the sequence of overall geometries assumed by the device 102′ during a venous transseptal procedure 600 (entering the right atrium 602 and then into the left atrium 608) is different from the sequence of overall geometries assumed by the device 102″ during a retrograde procedure 700 (moving through the descending (thoracic) aorta 702, into the left ventricle 606 and into the left atrium 608). In some embodiments the location of the device (as determined by one or more of magnetic, impedance, fiber optic localization systems) at different snapshots in time may be utilized to identify the procedure. In some embodiments, the locations are compared to anatomic models (e.g., 2D, 3D models of the heart) to detect the progression of the device. For example, with respect to the venous transeptal procedure 600 shown in FIG. 6, detection of the distal tip 120 in the right atrium 602 at a first point in time and then in the left atrium 608 at a subsequent point in time can be utilized to detect the type of procedure being utilized—in this case the venous transeptal procedure 600. In some embodiments, the detected overall geometries and/or path for a device may be compared to a database to identify the procedure. The database may include at least one procedure that may be performed, at least one overall geometry of the device the procedure, and/or a path travelled by the device during the procedure. In some embodiments, the procedure database may be stored locally on the data collection and analysis system 104 while in other embodiments the procedure database is stored remotely at the reprocess center 150. In some embodiments, device manipulations are detected based on the identification of the procedure being performed without requiring a measurement of the particular strain experienced by the device. Rather, a total usage capability or weighting is associated with each of the different procedures that may be detected. For example, the database may store a weighting value associated with each of the plurality of procedures that may be performed based on an expected amount of stress and/or strain encountered by the device during the procedure. For example, a device utilized for a venous transseptal procedure 600 typically encounters less stress than a device utilized for a retrograde procedure 700.

At step 210, the total usage capability of the device is updated based on the identification of the device utilized in the procedure, recorded device attributes, and detected device manipulations. In some embodiments, the total usage capability may include a number of attributes/manipulations analyzed individually or in combination. That is, the total usage capability may be numerical representation of the fusion of a plurality of different device attributes and/or device manipulations. For example, total usage capability may incorporate both time of use and detected device manipulations into the total usage capability. In other embodiments, each of the plurality of device attributes and/or detected device manipulations may be analyzed separately and then combined. For example, time of use may be compared to a threshold value and assigned a value based on the comparison. Likewise, the number and magnitude of catheter manipulations may be compared to a threshold value and assigned a value. The output of the plurality of comparisons may be combined to determine a total usage capability.

At step 212, the total usage capability is utilized to determine whether the device may be reprocessed. In some embodiments, the total usage capability starts at an initial maximum value and decreases as the device is utilized. For example, the total usage capability of the device may be initialized to a value of 100 and may decrease as the device is used. The threshold value for determining whether the device may be reprocesses may be zero or may be some arbitrary number greater than zero (e.g., 20). If the total usage capability is greater than a threshold value then the device is eligible to be reprocessed at step 214. If the total usage capability is less than the threshold value then the device is discarded/retired at step 216. The decrease in the total usage capability of the device may reflect a combination of factors, including total time the device has been in use, total number of device manipulations detected, and/or total number of ablation cycles initiated. In other embodiments, the total usage capability may be initialized to a value of zero and increase as the device is utilized. In this case, the device may be reprocessed if below a threshold value and discarded/retired if greater than a threshold value.

In other embodiments, the total usage capability is comprised of a plurality of comparisons, each of which must be true for the device to be reprocessed at step 214. For example, the total usage capability may require that the total hours of use be less than a threshold value, and that the total mechanical manipulations and magnitude of those manipulations must be less than a threshold number, and that the total number of ablative cycles delivered by the device is less than a threshold value. If each of these statements is true, then the device may be reprocessed at step 214. If one or more of these statements is false, then the device is discarded/retired at step 216.

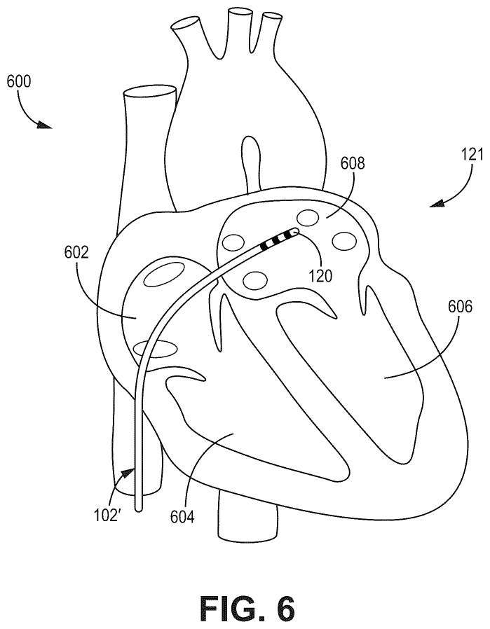

FIG. 3 is a flowchart of a method of determining device manipulations based on location information of sensors located on the device according to some embodiments. Reference is made to FIGS. 4A, 4B, and 5 to illustrate analysis of sensor location to detect device manipulations. FIGS. 4A and 4B are isometric views of a grid array 402 located at a distal end of catheter 400. FIG. 4A illustrates the grid-array 402 is a flexed state and FIG. 4B illustrates the grid-array 402 in an unflexed state. Flexing of the grid array 402 is a type of device manipulation that contributes to the total usage capability of the device. The grid array 402 includes a proximal end 406, a distal end 408, a plurality of arms 410a, 410b, 410c, and 410d, each arm including a plurality of ring electrodes 412a, 412b, 412c, and 412d, as well as two shaft electrodes 414a, 414b located on catheter shaft 404. During deployment/retraction from a distal end of an introducer, the grid array 402 may transition between a contracted state and an expanded state. Likewise, catheter 500 shown in FIG. 5 includes a loop portion 502, a shaft 504 having a distal end 506, a plurality of shaft electrodes 508a, 508b, and a plurality of ring electrodes 510a, 510b, 510c located on the loop portion 502. During deployment/retraction from a distal end of an introducer, loop portion 502 may transition between a contracted state and an expanded state. For example, the loop portion 502 may be substantially linear in the contracted state and substantially round (circular/oval) in the expanded state.

At step 302 shown in FIG. 3, location information is received from the plurality of sensors located on the catheter. For example, as shown in FIGS. 4a and 4b, location information is received with respect to electrodes 412a, 412b, 412c, and 412d, as well as shaft electrodes 414a and 414b.

In some embodiments, the location of electrodes is determined utilizing impedance-based localization. In other embodiments, location information may be received from other types of sensors, including magnetic-based sensors.

At step 304, distances are measured between respective sensors located on the catheter. For example, with reference to FIGS. 4a and 4b, a distance is measured between shaft electrode 414a and electrodes 410a and 410d. In FIG. 4a—in which the grid array 402 is in a flexed state—the measured distances are labeled d1 and d2. In FIG. 4b—in which the grid array 402 is not unflexed—the measured distances are labeled d3 and d4. As the grid array 402 is flexed, the distance between these respective electrodes decreases. For example, the distance d1 is less than the distance d3. Likewise, the distance d2 is less than the distance d4. This change in distance is utilized to detect catheter manipulations. In some embodiments, the location of the sensors is determined by mapping/localization system (e.g., impedance-based localization system, magnetic-based localization system, or other types of localization systems provided by software modules 132a, 132b, and/or 132). The relative distance between these sensors may likewise be determined by the local system (e.g., data collection and analysis systems 104, shown in FIG. 1), or by a remote system (e.g., at reprocess center 105). Similarly, the distance between electrodes in the catheter 500 shown in FIG. 5 may likewise be utilized to determine the state of the loop (i.e., straight, curved). For example, the distance d5 between shaft electrode 508a and ring electrode 510a may be measured.

At step 306, the relative distances measured between the plurality of sensors are utilized to detect catheter manipulations. In some embodiments, the measured distances are compared to threshold values to detect catheter manipulation. For example, the distances d1 and d2 may be compared to threshold values, wherein if the measured distances are less than a threshold value a determination is made that the grid array 402 is being flexed. In other embodiments, distances between a plurality of sensors are utilized to detect catheter manipulations. In some embodiments, catheter manipulations are identified as binary events (that is, the catheter is either being flexed or it is not). In other embodiments, various degrees of catheter manipulation are determined. For example, based on the relative distances the flexing of the grid array 402 shown in FIGS. 4a and 4b may be relatively minor or may be more severe. In some embodiments, catheter manipulations may be grouped or categorized according to the stress of the manipulation, with the more severe the stress the greater the impact on the total usage capability of the catheter. In some embodiments, statistical data may be collected with respect to location of the sensors as a result of various types of catheter manipulations, wherein the location of the plurality of sensors may be compared to the statistical dataset to detect the catheter manipulation most likely being experienced by the catheter.

Although the embodiments shown in FIGS. 4a, 4b, and 5 all utilized electrode sensors, in other embodiments other types of sensors may be utilized to detect catheter manipulations. For example, magnetic-based sensors and/or fiber-optic based sensors may be utilized to detect catheter manipulations. In some embodiments, the location of various types of sensors may be utilized in combination. For example, the distance between a magnetic-based sensor and an electrode may be utilized to detect catheter manipulations. In still other embodiments, operator manipulation of the catheter via the handle 114 (shown in FIG. 1) may be utilized to detect catheter manipulations, either alone or in combination with utilizing sensor location to detect catheter manipulations.

In this way, this disclosure describes a method that provides greater granularity and information regarding catheter use and allows for better decisions to be made regarding the reprocessing of catheters. The methods described herein may be used in conjunction with other methods of determining whether catheters may be reprocessed, including but not limited to visual inspections and total number of uses.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method of monitoring total usage capability of a medical device, the method comprising:
monitoring one or more device attributes during a procedure;
detecting device manipulations during the procedure based on the one or more device attributes; and
determining whether the medical device may be reprocessed for subsequent use based, at least in part, on the detected device manipulations.

2. The method of claim 1, wherein the one or more device attributes includes at least one of:
location data associated with the medical device during the procedure;
time of use of the device; or
a measure of ablation activation time.

3. The method of claim 2, wherein detecting device manipulations during the procedure includes analyzing sensor location to detect mechanical device manipulations including flexing and/or bending of the medical device.

4. The method of claim 3, wherein the medical device is a loop catheter including one or more sensors located on a loop portion of the distal end, and wherein sensor location of each sensor is analyzed to detect at least one of:
opening or closing of the loop;
engagement of the loop against tissue; or
deployment and retraction of the loop from within an introducer.

5. The method of claim 3, wherein the medical device is a grid array catheter including a distal end having a plurality of flexible arms each carrying one or more sensors, wherein sensor location is analyzed to detect flexing of the plurality of flexible arms.

6. The method of claim 1, wherein determining whether the medical device may be reprocessed for subsequent use based, at least in part, on the detected device manipulations includes calculating a total usage capability of the medical device.

7. The method of claim 1, wherein detecting device manipulations during the procedure includes identifying the procedure, wherein identifying the procedure includes analyzing sensor location to determine:
at least one overall geometry assumed by the medical device during the procedure; or
a path of the medical device.

8. A method of tracking device manipulations, the method comprising:
receiving location information from two or more sensors located on an medical device during a procedure;
utilizing the location information from the two or more sensors to detect device manipulations; and
storing the detected device manipulations.

9. The method of claim 8 wherein the two or more sensors are magnetic-based sensors, impedance-based sensors, or a combination thereof.

10. The method of claim 8, wherein utilizing the location information from the two or more sensors to detect device manipulations includes comparing a location of a first sensor to a location of a second sensor to detect device manipulations.

11. The method of claim 10, wherein the medical device is a loop catheter including a first sensor located on a loop portion of the loop catheter, wherein a location of the first sensor located on the loop portion is utilized to determine whether the loop is open or closed.

12. The method of claim 11, wherein the loop catheter includes a second sensor, wherein a location of the second sensor relative to the first sensor is utilized to determine whether the loop is open or closed, wherein the second sensor is located proximal to the loop portion or on the loop portion.

13. The method of claim 10, wherein the medical device is a grid array catheter having a plurality of flexible arms located at a distal end of the catheter, each flexible arm having at least one sensor, wherein a location of sensors are compared to one another to detect flexing of the grid array catheter.

14. The method of claim 8, wherein utilizing the location information from the two or more sensors to detect device manipulations includes identifying a procedure performed with the device by determining:
a path traversed by the device during the procedure; or
at least one overall geometry assumed by the device during the procedure.

15. A data process and analysis system configured to receive inputs from one or more sensors associated with a medical device, the data process and analysis system including a processor and memory for storing instructions executed by the processor to:
determine locations of the one or more sensors based on inputs received from the one or more sensors;
detect device manipulations based on the determined locations of the one or more sensors; and
determine whether the medical device may be reprocessed for subsequent use based, at least in part, on the detected device manipulations.

16. The system of claim 15, wherein detecting device manipulations includes analyzing sensor location to detect mechanical device manipulations including flexing and/or bending of the medical device.

17. The system of claim 15, wherein the system is further configured to measure time of use of the medical device, wherein determining whether the medical device may be reprocessed for subsequent use is further based on the time of use of the medical device.

18. The system of claim 17, wherein the system is further configured to measure ablation time of the medical device, wherein determining whether the medical device may be reprocessed for subsequent use is further based on the ablation time of the medical device.

19. The system of claim 15, wherein determining whether the medical device may be reprocessed includes calculating a total usage capability of the medical device.

20. The system of claim 15, wherein detecting device manipulations based on the determined locations of the one or more sensors includes identifying a procedure performed with the medical device by determining at least one of:
a path of the medical device; or
an overall geometry assumed by the medical device during the procedure.
